(19) United States
(12) Patent Application Publication
(10) Pub. No.: US 2019/0357556 A1
(43) Pub. Date: Nov. 28, 2019

(54) PRODUCTION METHOD FOR LOW-ACID FERMENTED MILK

(71) Applicant: MEIJI CO., LTD., Tokyo (JP)

(72) Inventors: Mari KASHIWAGI, Hachioji city, Tokyo (JP); Nobuko INOUE, Hachioji city, Tokyo (JP); Seiji NAGAOKA, Hachioji city, Tokyo (JP)

(21) Appl. No.: 16/486,497

(22) PCT Filed: Feb. 16, 2018
(86) PCT No.: PCT/JP2018/005466

§ 371 Date: Aug. 15, 2019

(30) Foreign Application Priority Data
Feb. 17, 2017 (JP) 2017-028318

(51) Int. Cl. A23C 9/123; C12N 1/20; C12R 1/225

(52) U.S. Cl. CPC A23C 9/1238; C12N 1/20; A23Y 2240/75; A23Y 2220/15; C12R 1/225

(57) ABSTRACT

To effectively suppress pH decrease during the second half of a fermentation process. This low-acid fermented milk production method comprises a step for obtaining a fermented milk base material by adding a lactobacilli starter to a raw material mix; and a fermentation step for fermenting the fermented milk base material at 35-50° C., wherein the time required for the pH of the fermented milk base material to drop from 4.6 to 4.4 in the fermentation step is set to three hours or more. Further, the time required for the pH of the fermented milk base material to reach 4.6 after the addition of the lactobacilli starter to the raw material mix is set to nine hours or less.

### TECHNICAL FIELD

The present invention relates to a production method of a fermented milk. Specifically, the present invention relates to a production method of a fermented milk in which the increase in sourness during fermentation is suppressed.

### BACKGROUND ART

A fermented milk is defined as, under “Ministerial Ordinance on Milk and Milk products Concerning Compositional Standards, etc.” (hereinafter referred to as “Ministerial Ordinance concerning Milk”) of Japan, a paste or a liquid, or a frozen product thereof obtained by subjecting milk or a milk-like substance or the like containing solids not fat in an amount equal to or more than that of milk to fermentation with a lactic acid bacterium or yeast. Examples of the fermented milk include a set type yogurt (a solid fermented milk), a soft type yogurt (a paste fermented milk), and a drink type yogurt (a liquid fermented milk). The set type yogurt is obtained mainly by filling a raw material mix into a container, then fermenting and solidifying the raw material mix in the container. The soft yogurt is obtained by fermenting a raw material mix, then crushing the curd, mixing it with pulp, sauce and the like as needed, and filling the mixture into a container. The drink yogurt is obtained by fermenting a raw material mix, then making it a liquid with a homogenizer and the like, mixing it with a sugar solution, a pulp source and the like as needed, and filling the mixture in a container.

Compositional Standards of Ministerial Ordinance concerning Milk of Japan stipulates that a fermented milk should have 8.0% or more of solids not fat and a total number of a lactic acid bacterium of at least 1.0×10⁷ cfu/g or more. Furthermore, the international standard of yogurt according to FAO/WHO also stipulates that the final product should have many live microorganisms (Lactobacillus bulgaricus and Streptococcus thermophilus).

Thus, a fermented milk contains many viable bacteria of a lactic acid bacterium and the like. When a fermented milk is stored for a long time at a fermentation promoting temperature range (for example, 30° C. to 50° C.), for example, to concentrate the fermented milk after pH of the fermented milk decreased to 4.6 or less at which the fermented milk generally have a stable structure, pH decreases due to lactic acid produced by lactic acid bacteria, and sourness increases. Thus, when a fermented milk is stored for a long time at a fermentation promoting temperature range, pH decreases over time compared to that immediately after the start of concentration, and thus, it has been considered difficult to keep the flavor and quality of the fermented milk constant for a long time.

Therefore, for example, a production method of highly palatable and highly concentrated yogurt is known in which milk is concentrated before fermentation, or milk component powder is added to milk to prepare a concentrated yogurt mix and then the mix is subjected to fermentation (Patent Literature 1). A production method of yogurt is also known in which the yogurt after fermentation is concentrated by membrane process or centrifugation to give richness to the yogurt (Patent Literature 2). As other methods, a method in which a lactic acid bacterium starter having low acid-producing capability is used can be mentioned.

### CITATION LIST

### Patent Literature

Patent Literature 1: JP 06-14707 A
Patent Literature 2: JP 2005-318855 A

### SUMMARY OF INVENTION

### Technical Problem

However, the method disclosed in Patent Literature 1 has a problem that the chalky taste, bitterness and saltiness are enhanced due to the mineral component derived from the milk material and the flavor of the milk component powder itself. In the method disclosed in Patent Literature 2, depending on the lactic acid bacterium starter used for fermentation, the sourness increases during the concentration step, and the palatability of the finally obtained concentrated yogurt may decrease.

Furthermore, when a lactic acid bacterium starter having low acid-producing capability is used, the fermentation time is long, which is unsuitable for industrial mass production, or the elapsed time for pH to decrease from 4.6 to 4.4 is short, and the degree of quality (especially sourness) varies if the product is not cooled quickly. That is, in the case of industrial mass production of a fermented milk, considering the production efficiency, it is desirable that the fermentation rate of the raw material mix be high (fermentation time be short) in the first half of fermentation from about pH 6.6 to pH 4.6. However, when the fermentation rate is high, lactic acid and the like are produced early. Thus, there is a problem that pH decreases more and the degree of sourness is increased in the second half of fermentation when the fermented milk is stored for a long time in the fermentation promoting temperature range (for example, 30° C. to 50° C.). For this problem, no technique has been proposed, which is capable of efficiently suppressing the decrease in pH in the second half of fermentation while maintaining the fermentation rate in the first half of fermentation at a rate suitable for mass production of a fermented milk.

The concentrated fermented milk in particular represented by Greek yogurt has a problem that the concentration step in which a fermented milk is separated into light liquid (whey) and heavy liquid (a concentrated fermented milk) by allowing the fermented milk to stand takes several hours, thus the fermentation further proceeds during the step, and the sourness of the finally obtained product is enhanced more. As a countermeasure against such a problem, in the concentration step, the fermentation can be suppressed by lowering the temperature of the fermented milk before concentration. However, the efficiency of separation into the light liquid and the heavy liquid is extremely decreased. As other countermeasures, there is also a method in which a lactic acid bacterium starter having low acid-producing capability is used. However, the method has a problem that the fermentation time is long, which is not suitable for industrial production of a concentrated fermented milk. Therefore, considering industrial production of a concentrated fermented milk, it is desirable to use a lactic acid bacterium starter having a certain acid-producing capability such as Lactobacillus bulgaricus and Streptococcus thermophilus and to maintain the temperature of the fermented milk at around 40° C. in the concentration step. However, under such a condition, the fermentation of the fermented milk proceeds in the concentration step, and the sourness of the final product (the concentrated fermented milk) also increases. Thus, it is considered difficult to suppress the sourness and fermentation odor of the concentrated fermented milk.

Therefore, basically, it is an object of the present invention to propose a production method of a fermented milk and a strain of a lactic acid bacterium starter that are capable of effectively suppressing the decrease in pH, in particular, in the second half of fermentation. It is also an object of the present invention, for example in the production of a concentrated fermented milk, to obtain a concentrated fermented milk in which the sourness and the fermentation odor are suppressed by using a lactic acid bacterium starter having a certain acid-producing capability such as Lactobacillus bulgaricus and Streptococcus thermophilus and suppressing the decrease in pH in concentration step even when the temperature of the fermented milk is maintained at around 40° C. in the concentration step.

### Solution to Problem

A first aspect of the present invention relates to a production method of a fermented milk. The production method according to the present invention includes a step for adding a lactic acid bacterium starter to a raw material mix to obtain a fermented milk base material, and a fermentation step for fermenting the fermented milk base material at 35° C. to 50° C. In the fermentation step, a time required for pH of the fermented milk base material to decrease from 4.6 to 4.4 is 3 hours or more.

As described above, when the time required for pH of the fermented milk base material to decrease from 4.6 to 4.4 is 3 hours or more, for example, even in the production of a concentrated fermented milk, a concentrated fermented milk in which the sourness and fermentation odor are suppressed can be obtained by suppressing the decrease in pH in the concentration step. The present invention can be applied to the production method of a fermented milk in general, and is not limited to the production method of a concentrated fermented milk.

In the production method of a fermented milk according to the present invention, a time required for pH of the fermented milk base material to reach 4.6 after addition of the lactic acid bacterium starter to the raw material mix is preferably within 9 hours. The time required for pH to reach 4.6 is more preferably 8 hours or less, and particularly preferably 7.5 hours or less.

As described above, when the time required for pH to reach 4.6 after the completion of inoculation of the lactic acid bacterium starter is 9 hours or less, decrease in production efficiency of a fermented milk can be avoided. That is, the decrease in pH in the second half of fermentation can be effectively suppressed while maintaining the fermentation rate of the fermented milk in the first half of fermentation. Specifically, since the cooling condition of the fermented milk can be relaxed, equipment investment can be reduced and energy saving can be achieved. The quality deterioration due to over-fermentation of the fermented milk can be suppressed even if problems and the like occur in the production. The decrease in viscosity and the decrease in the number of lactic acid bacteria due to excessive rapid cooling can be prevented. Furthermore, in the case of producing a concentrated fermented milk such as Greek yogurt, a product in which the sourness is suppressed can be prepared under production conditions with high concentration efficiency (for example, fermentation temperature of 35° C. to 50° C.).

In the production method of a fermented milk according to the present invention, a lactic acid bacterium starter including Lactobacillus bulgaricus and Streptococcus thermophilus having a particular bacteriological property is preferably used. Specifically, the Lactobacillus bulgaricus and Streptococcus thermophilus preferably have a bacteriological property that changes acidity of lactic acid of a skimmed milk powder medium containing 0.1 wt % of yeast extract to 0.8 or more and less than 1.0 when the Lactobacillus bulgaricus and Streptococcus thermophilus are each purely cultured in the skimmed milk powder medium at 37° C. to 43° C. for 12 hours. Furthermore, the Lactobacillus bulgaricus and Streptococcus thermophilus preferably have a bacteriological property that changes pH of the medium to 4.1 or more and 4.6 or less under the same measurement conditions. The lactic acid bacterium starter can consist only of the above-mentioned Lactobacillus bulgaricus and Streptococcus thermophilus.

As described above, by selecting strains that satisfy the above-mentioned culture conditions from among strains of the Lactobacillus bulgaricus and Streptococcus thermophilus and inoculating them into the raw material mix, the time required for pH of the fermented milk base material to decrease from 4.6 to 4.4 becomes 3 hours or more while maintaining the time required for pH to reach 4.6 after the completion of inoculation of the lactic acid bacterium starter at 9 hours or less. That is, when the acidity of lactic acid after the above-mentioned pure culture is 0.80 or more, the time required for pH to reach 4.6 from the start of fermentation becomes shorter, and productivity can be further increased. When the acidity of lactic acid after the above-mentioned pure culture is less than 1.0, the decrease in pH (increase in acidity) in the second half of fermentation can be more effectively suppressed. In the present invention, when the Lactobacillus bulgaricus and Streptococcus thermophilus of particular strains are used as a lactic acid bacterium starter, the decrease in pH of the fermented milk in the second half of fermentation can be suppressed, thus no special treatment is required to be performed in the fermentation step, and productivity for mass production of the fermented milk can be maintained.

In the production method of a fermented milk according to the present invention, the combination of a strain of the Lactobacillus bulgaricus and a strain of the Streptococcus thermophilus included in the lactic acid bacterium starter is preferably selected from a combination of a strain of the Lactobacillus bulgaricus and a strain of the Streptococcus thermophilus that decreases pH to 4.6 or less within 9 hours when the combination is subjected to mixed culture in a skimmed milk powder medium at 37° C. to 43° C. The lactic acid bacterium starter can consist only of such a combination of a strain of the Lactobacillus bulgaricus and a strain of the Streptococcus thermophilus.

In the production method of a fermented milk according to the present invention, the Lactobacillus bulgaricus is preferably a Lactobacillus delbrueckii subsp. bulgaricus OLL205013 strain (Accession No.: NITE BP-02411). The Streptococcus thermophilus is preferably a Streptococcus thermophilus OLS3290 strain (Accession No.: FERM BP-19638) or OLS3615 strain (Accession No.: NITE BP-01696). In particular, it is preferable that Bulgaria be an OLL205013 strain, and the Streptococcus thermophilus be an OLS3290 strain. The lactic acid bacterium starter consists of a combination of these particular strains of the Lactobacillus bulgaricus and Streptococcus thermophilus.

As shown in Examples described below, the present inventors have found that the effect of the present invention can be exhibited more remarkably by using a lactic acid bacterium starter in which a Lactobacillus delbrueckii subsp. bulgaricus OLL205013 strain and a Streptococcus thermophilus OLS3290 strain (or OLS3615 strain) are combined.

A second aspect of the present invention relates to a strain of a lactic acid bacterium (Lactobacillus bulgaricus) included in a lactic acid bacterium starter. The lactic acid bacterium of the present invention is a Lactobacillus delbrueckii subsp. bulgaricus OLL205013 strain (Accession No.: NITE BP-02411). The OLL205013 strain has the following bacteriological properties.

a) The OLL205013 strain changes the acidity of lactic acid of a skimmed milk powder medium containing 0.1 wt % of yeast extract to 0.8 or more and less than 1.0 when the strain is purely cultured in the skimmed milk powder medium at 37° C. to 43° C. for 12 hours.

b) The OLL205013 strain changes the pH of a skimmed milk powder medium containing 0.1 wt % of yeast extract to 4.1 or more and 4.6 or less when the strain is purely cultured in the skimmed milk powder medium at 37° C. to 43° C. for 12 hours.

c) The OLL205013 strain decreases pH to 4.6 or less within 9 hours when the strain is subjected to mixed culture with other strains of Streptococcus thermophilus in a skimmed milk powder medium at 37° C. to 43° C.

As shown in the Examples described below, the present inventors have found that, in general, the fermentation rate in the first half of fermentation can be increased and the decrease in pH in the second half of fermentation can be suppressed by using the above-mentioned OLL205013 strain. That is, the study of the present inventors has proved that, in the production of a starter for fermented milk production in which Lactobacillus bulgaricus and Streptococcus thermophilus are mixed, when an OLL205013 strain is used as Lactobacillus bulgaricus, the decrease in pH in the second half of fermentation can be suppressed while increasing the fermentation rate in the first half of fermentation, regardless of what strain is used as Streptococcus thermophilus to some extent. Therefore, it is presumed that the OLL205013 strain is at the core of the characteristic effect of the present invention.

### Advantageous Effects of Invention

The present invention provides a production method of a fermented milk and a lactic acid bacterium starter capable of effectively suppressing the decrease in pH in the second half of fermentation.

### DESCRIPTION OF EMBODIMENTS

Hereinafter, embodiments for carrying out the present invention will be described with reference to the drawings. The present invention is not limited to the embodiments described below, but includes modifications from the following embodiments appropriately made by those skilled in the art within the obvious scope.

In the description of the present application, “Accession No.: FERM . . . ” means Accession No. at the National Institute of Advanced Industrial Science and Technology, International Patent Organism Depositary, which is an international depositary authority under the Budapest Treaty. “Accession No.: NITE . . . ” means Accession No. at National Institute of Technology and Evaluation, Patent Microorganisms Depositary, which is an international depositary authority under the Budapest Treaty.

In the description of the present application, “A to B” means “A or more and B or less” unless otherwise specified.

In the description of the present application, “raw material mix” is a liquid containing a milk component such as raw milk, whole fat milk, skimmed milk, and whey, and means those before the starter addition step. Here, the raw milk refers to, for example, animal milk such as cow's milk. The raw material mix can include, in addition to a milk component such as raw milk, whole fat milk, skimmed milk, and whey, processed products thereof (for example, whole fat milk powder, whole fat concentrated milk, skimmed milk powder, skimmed milk concentrate, condensed milk, whey powder, buttermilk, butter, cream, cheese, whey protein concentrate (WPC), whey protein isolate (WPI), α-lactalbumin (α-La), β-lactoglobulin (β-Lg) and the like). “Fermented milk base material (yogurt base)” means the raw material mix after the addition of a lactic acid bacterium starter. “Fermented milk” means the resulting product after the completion of the fermentation step that is obtained by fermenting a fermented milk base material.

The present invention relates to a production method of a fermented milk. An example of the fermented milk is yogurt. The fermented milk can be a set type yogurt, a soft type yogurt, or a drink type yogurt. The fermented milk produced according to the present invention can be used as a material for a frozen yogurt. The fermented milk produced according to the present invention can be used as a material for a cheese. In the present invention, the fermented milk can be any of “fermented milk”, “dairy product lactic acid bacterium beverage”, “lactic acid bacterium beverage” and the like defined by Ministerial Ordinance concerning Milk.

The production method of a fermented milk according to the present invention basically includes a raw material mix preparation step, a heat sterilization step, a primary cooling step, a starter addition step, a heating step, a fermentation step, and a secondary cooling step.

In the production of a fermented milk, firstly, the raw material mix preparation step is performed. The raw material mix preparation step is a step of preparing a raw material mix used as a material of a fermented milk. The raw material mix is also called a yogurt mix. In the present invention, a known raw material mix can be used. For example, the raw material mix can be composed of only raw milk (100% raw milk). The raw material mix can be prepared by mixing, in addition to a milk component such as raw milk, whole fat milk, skimmed milk, and whey, processed products thereof (for example, whole fat milk powder, whole fat concentrated milk, skimmed milk powder, skimmed milk concentrate, condensed milk, whey powder, buttermilk, butter, cream, cheese, whey protein concentrate (WPC), whey protein isolate (WPI), α-lactalbumin (α-La), β-lactoglobulin (β-Lg) and the like). The raw material mix can include, in addition to a milk component, a food, a food composition, a food additive and the like such as soy milk, sugar, a saccharide, a sweetener, a flavor, fruit juice, pulp, a vitamin, a mineral, a fat and oil, a ceramide, collagen, a milk phospholipid, and a polyphenol. The raw material mix can include a stabilizer, a thickener, and a gelling agent or the like such as pectin, soybean polysaccharide, CMC (carboxymethylcellulose), agar, gelatin, carrageenan, and a gum, as needed. In the raw material mix preparation step, fat particles and the like included in the raw material mix are preferably atomized (ground) by a homogenization step in which the raw material mix is homogenized. This homogenization step can suppress or prevent the separation and surfacing of fats of the raw material mix, the fermented milk base material, and the fermented milk during or after the production step of the fermented milk.

The heat sterilization step is performed after the raw material mix preparation step. The heat sterilization step is a step of heating and sterilizing the raw material mix. For example, in the heat sterilization step, the raw material mix can be heat-treated at an adjusted heating temperature and heating time to the extent that bacteria in the raw material mix are sterilized. In the present invention, for the heat sterilization step, a known method can be used. For example, in the heat sterilization step, heat treatment can be performed with a plate heat exchanger, a tube heat exchanger, a steam injection heater, a steam infusion heater, a current heater and the like, or can be performed with a jacketed tank. In the heat sterilization step, when the yogurt is of a plain type, a hard type, or a soft type, heat treatment such as high temperature short time sterilization treatment (HTST) can be performed, and when the yogurt is of a drink type, heat treatment such as ultra high temperature sterilization treatment (UHT) can be performed. For example, in the heat sterilization step, the high temperature short time sterilization treatment (HTST) can be a treatment in which the raw material mix is heated to 80° C. to 100° C. for about 3 minutes to 15 minutes, and the ultra high temperature sterilization treatment (UHT) can be a treatment in which the raw material mix is heated to 110° C. to 150° C. for about 1 second to 30 seconds.

The primary cooling step is performed after the heat sterilization step. The primary cooling step is a step of cooling the heat-sterilized raw material mix to a predetermined temperature. In the primary cooling step, the raw material mix is cooled to a temperature lower than the fermentation promoting temperature range (for example, 30° C. to 50° C.). In the present invention, for the primary cooling step, a known method can be used. For example, in the primary cooling step, the cooling treatment can be performed with a plate heat exchanger, a tube heat exchanger, or a vacuum (decompression) evaporative cooler, or can be performed with a jacketed tank. Specifically, in the primary cooling step, the raw material mix is preferably cooled to 15° C. or less. In the primary cooling step, the raw material mix is preferably cooled to 1° C. to 15° C., more preferably cooled to 3° C. to 10° C., and further preferably cooled to 5° C. to 8° C.

In the primary cooling step, it is preferable that the raw material mix having a temperature increased in the heat sterilization step, around 100° C., be rapidly cooled to a low temperature (15° C. or less). For example, in the primary cooling step, when the sterilization step is heat treatment, the time to cool the raw material mix having a temperature increased in the sterilization step, around 100° C., to 15° C. is preferably within 10 minutes, more preferably within 5 minutes, further preferably within 1 minute, and particularly preferably within 30 seconds. This cooling step can suppress and prevent excessive denaturation of proteins and browning of carbohydrates in the raw material mix.

The starter addition step is performed after or during the cooling step. The starter addition step is a step of adding (mixing) a lactic acid bacterium starter to the raw material mix to obtain a fermented milk base material. That is, the lactic acid bacterium starter can be added after the temperature of the raw material mix decreased to a predetermined temperature after the heat sterilization step, or the lactic acid bacterium starter can be added while the temperature of the raw material mix is decreasing to a predetermined temperature after the heat sterilization step. In the present invention, for the starter addition step, a known method can be used. However, in the present invention, it is preferable that the lactic acid bacterium starter include at least Lactobacillus bulgaricus and Streptococcus thermophilus. That is, “Lactobacillus bulgaricus” is Lactobacillus delbrueckii subsp. bulgaricus, and “Streptococcus thermophilus” is Streptococcus thermophilus. In the present invention, in the starter addition step, in addition to Lactobacillus bulgaricus and Streptococcus thermophilus, a known lactic acid bacterium can be added (mixed). For example, in the starter addition step, Gasseri bacteria (Lactobacillus gasseri), Lactis bacteria (Lactococcus lactis), Cremoris bacteria (Lactococcus cremoris), Bifidus bacteria (Bifidobacterium and the like) can be added (mixed). It is particularly preferable that the lactic acid bacterium starter consist only of Lactobacillus bulgaricus and Streptococcus thermophilus as lactic acid bacteria. Meanwhile, the addition amount of a lactic acid bacterium starter can be a quantity employed in a known production method of a fermented milk.

In the present invention, the Lactobacillus bulgaricus and Streptococcus thermophilus included in the lactic acid bacterium starter preferably have a property that changes acidity of lactic acid of a skimmed milk powder medium containing 0.1 wt % of yeast extract to 0.8 or more and less than 1.0 (excluding 1.0) when the Lactobacillus bulgaricus and Streptococcus thermophilus are purely cultured in the medium at 37° C. to 43° C. for 12 hours (hereinafter called “a first property”). In particular, the acidity of lactic acid of the medium is preferably changed to 0.8 to 0.98, and more preferably changed to 0.8 to 0.95 under the same conditions. The “skimmed milk powder medium” is a medium consisting of skimmed milk powder and water, and means, in particular, a medium consisting of 10 wt of skimmed milk powder and 90 wt % of water. The “yeast extract” is specifically brewer's yeast extract, and is contained in the skimmed milk powder medium in an amount of 0.1 wt % relative to 100 wt % of the skimmed milk powder medium. “Pure culture” is a culture method in which one type of a lactic acid bacterium is cultured in one medium in a state where Lactobacillus bulgaricus and the Streptococcus thermophilus are separated. In the description of the present application, “acidity” (acidity of lactic acid) of the medium is measured according to the “Test Method for Compositional Standards on Milk and Milk Products” under the Ministerial Ordinance concerning Milk Specifically, 10 ml of ion-exchanged water containing no carbon dioxide gas is added to 10 g of the sample, and then 0.5 ml of phenolphthalein solution is added thereto as an indicator. Then, while adding a sodium hydroxide solution (0.1 mol/L), the sample is titrated using the point in which the pink color disappears as a limit, and the content of lactic acid per 100 g of the sample is calculated from the titer of the sodium hydroxide solution to obtain the acidity (acidity of lactic acid). The phenolphthalein solution is prepared by dissolving 1 g of phenolphthalein in an ethanol solution (50%) and filling up the resulting solution to 100 ml.

Furthermore, in the present invention, the combination of a strain of the Lactobacillus bulgaricus and a strain of the Streptococcus thermophilus is preferably selected from a combination of a strain of the Lactobacillus bulgaricus and a strain of the Streptococcus thermophilus having a property that decreases pH to 4.6 or less within 9 hours when the combination is subjected to mixed culture in a skimmed milk powder medium at 37° C. to 43° C. (hereinafter called “a second property”). “Mixed culture” is a culture method in which both types of lactic acid bacteria are cultured in one medium in a state where Lactobacillus bulgaricus and Streptococcus thermophilus are mixed. In the description of the present application, “pH” is measured according to the following method. That is, using a glass electrode type pH meter (HM-30R, manufactured by DKK-TOA CORPORATION, equipped with a temperature calibration function), a glass electrode is inserted into 100 g of sample, the measurement is read when the value becomes constant, and is taken as the pH of the sample.

In the present invention, the lactic acid bacterium starter having the above-described first property and the second property in which Lactobacillus bulgaricus and Streptococcus thermophilus are mixed is preferably used in the production of a fermented milk. Thereby, as shown in the Examples described below, the time required for pH of the fermented milk base material to decrease from 4.6 to 4.4 (the second half of fermentation) becomes 3 hours or more while maintaining the time required for pH of the fermented milk base material to reach 4.6 after addition of the lactic acid bacterium starter to the raw material mix (the first half of fermentation) within 9 hours.

Examples of Lactobacillus bulgaricus having the above-described first and second properties include Lactobacillus delbrueckii subsp. bulgaricus OLL205013 (Accession No.: NITE BP-02411). Examples of Streptococcus thermophilus having the above-described first and second properties include a Streptococcus thermophilus OLS3290 strain (Accession No.: FERM BP-19638) and an OLS3615 strain (Accession No.: NITE BP-01696). Therefore, in the present invention, as the lactic acid bacterium starter, a mixture of an OLL205013, Lactobacillus bulgaricus, and an OLS3290 strain or OLS3615 strain, Streptococcus thermophilus, is preferably used. In particular, when the OLL205013 is selected as Lactobacillus bulgaricus and the OLS3290 strain is selected as a Streptococcus thermophilus, the effects of the present invention are more remarkably exhibited.

In the starter addition step, the numbers of bacteria (viable cell count) of Lactobacillus bulgaricus and Streptococcus thermophilus contained in the lactic acid bacterium starter can be numbers employed in a known production method of a fermented milk. For example, the ratio of the number of bacteria of Lactobacillus bulgaricus to the number of bacteria of Streptococcus thermophilus contained in the lactic acid bacterium starter is generally 1:4 to 1:5. Specifically, in the starter addition step, the ratio of the number of bacteria of Lactobacillus bulgaricus relative to the number of bacteria of Streptococcus thermophilus contained in the lactic acid bacterium starter (standard), 1, (the number of bacteria of Lactobacillus bulgaricus/the number of bacteria of Streptococcus thermophilus) can be 0.01 to 0.8, is preferably 0.05 to 0.7, more preferably 0.1 to 0.5, and further preferably 0.2 to 0.4. Meanwhile, in the starter addition step, for the number of bacteria of Lactobacillus bulgaricus and the number of bacteria of Streptococcus thermophilus (viable cell count) contained in the lactic acid bacterium starter, the lactic acid bacterium starter can contain a larger number of bacteria of Lactobacillus bulgaricus than the number of bacteria of Streptococcus thermophilus in advance. For example, the ratio of the number of bacteria of Lactobacillus bulgaricus relative to the number of bacteria of Streptococcus thermophilus contained in the lactic acid bacterium starter can be 1.0 to 5.0, 1.5 to 4.0 or the like. The number of bacteria of a lactic acid bacterium can be measured according to a known method.

The heating step is performed after the starter addition step. The heating step is a step of heating the fermented milk base material that has been cooled to the extent that the lactic acid bacterium starter can be added (1° C. to 15° C.) to a fermentation promoting temperature range (for example, 30° C. to 50° C.). The “fermentation promoting temperature range” means a temperature at which a microorganism (such as a lactic acid bacterium) is activated to advance or promote the fermentation of the fermented milk base material. In the present invention, for the heating step, a known method can be used. For example, in the heating step, the heat treatment can be performed with a plate heat exchanger, a tube heat exchanger, or the like, or the heat treatment can be performed with a jacketed tank. For example, the fermentation promoting temperature range of the lactic acid bacterium is generally 30° C. to 50° C. Specifically, in the heating step, the fermented milk base material is preferably heated to 30° C. or more. Furthermore, in the heating step, the fermented milk base material is preferably heated to 30° C. to 50° C., more preferably 33° C. to 48° C., and further preferably 35° C. to 46° C.

Further, in the heating step, the fermented milk base material whose temperature has been decreased in the primary cooling step is preferably heated to a fermentation promoting temperature range within a predetermined time (in a relatively short time). For example, in the heating step, the time of heating the fermented milk base material at about 10° C. whose temperature has been decreased in the low temperature holding step to the fermentation promoting temperature range is preferably within 1 hour, preferably within 30 minutes, further preferably within 10 minutes, and particularly preferably within 1 minute. In the heating step, the heat treatment can be performed by transferring the fermented milk base material whose temperature has been decreased as it is to a fermentation chamber set to room temperature of about 30° C. to 50° C., and gradually heating the fermented milk base material.

The fermentation step is performed after the heating step. The fermentation step is a step of fermenting the fermented milk base material heated to the fermentation promoting temperature range with the fermented milk base material held at the fermentation promoting temperature range. Specifically, the fermentation of the fermented milk base material is performed in a temperature range of 35° C. to 50° C. For the fermentation step, a known method can be used. For example, in the fermentation step, the fermentation treatment can be performed in a fermentation chamber or the like, or the fermentation treatment can be performed in a jacketed tank. Furthermore, for example, in the fermentation step, the fermented milk base material can be fermented by maintaining the temperature of the fermented milk base material at 35° C. to 50° C. in a fermentation chamber where the temperature (the fermentation temperature) is maintained at 30° C. to 50° C. In the fermentation step, the fermented milk base material can be also fermented by maintaining the temperature of the fermented milk base material at 35° C. to 50° C. in a jacketed tank where the temperature (the fermentation temperature) is maintained at 30° C. to 50° C. In the fermentation step, for the conditions of fermenting the fermented milk base material, the fermentation temperature, the fermentation time and the like can be appropriately adjusted in consideration of type and quantity of the raw material mix and lactic acid bacteria, the flavor, mouthfeel and the like of the fermented milk. Specifically, in the fermentation step, the fermented milk base material is preferably held at 35° C. or more. Furthermore, in the fermentation step, the fermented milk base material is preferably held at 35° C. to 50° C., more preferably held at 37° C. to 48° C., and particularly preferably held at 40 to 46° C. Specifically, in the fermentation step, the fermented milk base material is preferably held at the fermentation promoting temperature range for 1 hour or more. In the fermentation step, the period of holding the fermented milk base material (the fermentation time) is preferably 3 to 30 hours, more preferably 6 to 25 hours, and further preferably 10 to 20 hours. In the present invention, the temperature of the fermented milk base material during the fermentation step can be maintained constant in the range of 35° C. to 50° C., and there is no need to increase or decrease the temperature.

The fermentation step includes the first half of fermentation and the second half of fermentation. The first half of fermentation is a period from when the lactic acid bacterium starter is added to the raw material mix until the pH of the fermented milk base material reaches 4.6. It can be said that the shorter the time of the first half of fermentation, the higher the production efficiency of the fermented milk. In the present invention, under the temperature conditions where the temperature of the fermented milk base material is maintained at 35° C. to 50° C., the time required for the first half of fermentation is within 9 hours. The time required for the first half of fermentation is preferably within 8 hours, and more preferably within 7 hours. Though the lower limit of the time required for the first half of fermentation is not particularly limited, it is preferably, for example, 4 hours or more, 5 hours or more, or 6 hours or more.

The second half of fermentation is a period of the pH of the fermented milk base material decreasing from 4.6 to 4.4. It can be said that the longer the second half of fermentation, the less the variation in the quality (especially acidity) even when the fermented milk is held for a long time at a fermentation promoting temperature range (for example, 30° C. to 50° C.). In the present invention, under the temperature conditions where the temperature of the fermented milk base material is maintained at 35° C. to 50° C., the time required for the second half of fermentation is 3 hours or more. The time required for the second half of fermentation is preferably 3.5 hours or more, more preferably 4 hours or more, and further preferably 4.5 hours or more. Though the upper limit of the time required for the second half of fermentation is not particularly limited, it is preferably, for example, 10 hours or less, 8 hours or less, or 6 hours or less.

According to the present invention, in the fermentation step, it is possible to ferment the fermented milk base material for a long time while suppressing the increase in sourness of the fermented milk base material. Therefore, the present invention is suitable for producing a concentrated fermented milk with suppressed sourness. Therefore, in the fermentation step, a concentration step can be performed in which the fermented milk base material is allowed to stand to separate the fermented milk base material into light liquid (whey) and heavy liquid (a concentrated fermented milk). After the separation step, a fermented milk (a concentrated fermented milk) in which milk components are concentrated can be obtained by removing the light liquid from the fermented milk base material. The term “allowing to stand” as used herein means to leave the fermented milk base material calmly without applying external pressure to an extent that the fermented milk base material can be separated into light liquid with a light mass and heavy liquid with a heavy mass under a natural condition without stirring or mixing. When such a concentration step is performed, it is preferable that the temperature of the fermented milk base material in the fermentation step be in the fermentation promoting temperature range of 30° C. to 50° C. (preferably 35° C. to 50° C.), and the fermentation time be 9 hours or more (preferably 10 hours or more). Though the temperature of the fermented milk base material can be decreased to, for example, 10° C. or less in the fermentation step, this is not preferable because the speed of separation between the light liquid and the heavy liquid becomes extremely slow. In the present invention, the concentration step is not a necessary step, and producing a usual fermented milk (yogurt) that does not undergo the concentration step is also possible.

The secondary cooling step is performed after the fermentation step. The secondary cooling step is a step of cooling the fermented milk (in particular, the concentrated fermented milk) obtained in the fermentation step. In the secondary cooling step, the progress of the fermentation is suppressed by decreasing the temperature of the fermented milk. At this time, in the secondary cooling step, the fermented milk is cooled to a temperature lower than the fermentation promoting temperature range. In the present invention, for the secondary cooling step, a known method can be used. For example, in the secondary cooling step, the cooling treatment can be performed with a refrigerator or a freezer, or the cooling treatment can be performed with a plate heat exchanger, a tube heat exchanger, or a jacketed tank. Specifically, in the secondary cooling step, the fermented milk is preferably cooled to 15° C. or less. In the secondary cooling step, the fermented milk is preferably cooled to 1° C. to 15° C., more preferably cooled to 3° C. to 10° C., and further preferably cooled to 5° C. to 8° C. In this secondary cooling step, cooling the fermented milk to a temperature suitable for food suppresses and prevents changes in flavor (sourness and the like), mouthfeel (feeling on the tongue and the like), and physical properties (hardness and the like) of the fermented milk. The fermented milk after the secondary cooling step can be stored in a refrigerator or the like for a long time at a low temperature of 3° C. to 10° C.

### EXAMPLES

Hereinafter, the present invention will be specifically described with Examples. However, the present invention is not limited to the following Examples, and various improvements based on known methods can be added.

[Preparation of Mother Starter]

A skimmed milk powder medium in which skimmed milk powder: 10 wt %, brewer's yeast: 0.1 wt %, and water: 89.9 wt % are mixed was sterilized at 121° C. for 7 minutes, and then cooled to room temperature. In this medium, various strains of Lactobacillus bulgaricus and Streptococcus thermophilus were subjected to activation culture three times. Then, 1 wt % of each of the various strains after the activation culture was inoculated into another skimmed milk powder medium prepared in the same way as above, and purely cultured for 12 hours at 37° C. to obtain a mother starter. As Lactobacillus bulgaricus, an OLL1222 strain, an OLL205013 strain (Accession No.: NITE BP-02411), and an OLL1171 strain (Accession No.: NITE BP-01569) were cultured, and as Streptococcus thermophilus, a 203P1 strain, an OLS3290 strain (Accession No.: FERM BP-19638), and an OLS3615 strain (Accession No.: NITE BP-01696), and a 203P2 strain were cultured. To examine the fermentability of the pure culture for various strains, 1 wt % of each of the mother starters was inoculated into the above-mentioned medium and purely cultured at 37° C. for 12 hours. The measurement results of the acidity and pH of the medium after the pure culture are shown in Table 1 below.

TABLE 1
Pure culture (12 hours)

| | L. bulgaricus OLL | | | S. thermophilus OLS | | | |
|---|---|---|---|---|---|---|---|
| | 1222 | 205013 | 1171 | 203P1 | 3290 | 3615 | 203P2 |
| Acidity | 0.94 | 0.86 | 1.00 * | 0.95 | 0.83 | 0.80 | 0.92 |
| pH | 4.17 | 4.31 | 4.00 * | 4.27 | 4.48 | 4.57 | 4.32 |

[Preparation of Bulk Starter]

A skimmed milk powder medium in which skimmed milk powder: 10 wt % and water: 90 wt % are mixed was heat-sterilized, and then cooled to 37° C. to prepare a bulk base. Into this bulk base, 1 wt % of one type of mother starters of Bulgaria and 1 wt % of one type of mother starters of Streptococcus thermophilus shown in Table 1 above were inoculated and then mixed. Then, the bulk base was cultured at 37° C. until the pH of the bulk base reached 4.5 or less, and then cooled to 5° C. to obtain a bulk starter.

[Preparation of Yogurt]

Skimmed milk powder: 10 wt % and water: 90 wt % were mixed, heated to 95° C. (sterilization), and then cooled to 10° C. to prepare a yogurt base. Into the yogurt base, 2 wt % of the above-mentioned bulk starter (the mixed starter of Lactobacillus bulgaricus and Streptococcus thermophilus) was inoculated and the yogurt base was fermented at 43° C. The times required for pH to reach 4.6 from the start of fermentation (at the time of inoculation of the bulk starter) (fermentation time) at that time are shown in Table 2 below. The times required for pH to decrease from 4.6 to 4.4 are shown in Table 3 below.

TABLE 2
The first half of fermentation: time required
for pH to reach 4.60 (minute)

| | | S. thermophilus OLS | | | |
|---|---|---|---|---|---|
| | | 203P1 | 3290 | 3615 | 203P2 |
| L. bulgaricus | 1222 | 428 | 388 | 448 | 384 |
| OLL | 205013 | 462 | 424 | 442 | 384 |
| | 1171 | — | — | 186 * | — |

TABLE 3
The second half of fermentation: time required
for pH to decrease from 4.60 to 4.40 (minute)

| | | S. thermophilus OLS | | | |
|---|---|---|---|---|---|
| | | 203P1 | 3290 | 3615 | 203P2 |
| L. bulgaricus | 1222 | 190 | 278 | 272 | 212 |
| OLL | 205013 | 210 | 294 | 252 | 206 |
| | 1171 | — | — | 50 * | — |

(minute)

In Table 3, the longer the time required for pH to decrease from 4.6 to 4.4 (the time required for the second half of fermentation), the more preferable it is. The time required for pH to decrease from 4.6 to 4.4 is required to be at least 3 hours or more. In the examples in which the OLL1222 strain and the OLL205013 strain of Lactobacillus bulgaricus were used, the times required for the second half of fermentation were 3 hours or more irrespective of the strain of Streptococcus thermophilus that was mixed with Lactobacillus bulgaricus, a 203P1 strain, an OLS3290 strain, an OLS3615 strain, and an 203P1 strain. Meanwhile, when a mixed starter of an OLL1171 strain of Lactobacillus bulgaricus and an OLS3615 strain of Streptococcus thermophilus was used, the time required for the second half of fermentation was less than 1 hour (specifically, 50 minutes), and it was impossible to extend the time. Therefore, it can be said that using the OLL1171 strain as Lactobacillus bulgaricus is not preferable to obtain a long time required for the second half of fermentation. In the above-mentioned Tables, data as a comparative example is indicated with “*”.

In Table 2, the shorter the time required for pH to reach 4.6 from the start of fermentation (the time required for the first half of fermentation), the more preferable it is. The time required for pH to reach 4.6 from the start of fermentation is required to be within 9 hours. In the data shown in Table 2, all the combinations had a time required for the first half of fermentation of within 9 hours. However, as shown in Table 3, the mixed starter of the OLL1171 strain of Lactobacillus bulgaricus and the OLS3615 strain of Streptococcus thermophilus was considered to be unsuitable because the time required for the second half of fermentation was short.

In Table 2 and Table 3, please see the mixed starter of the OLL205013 strain and the OLS3290 strain, and the mixed starter of the OLL205013 strain and the OLS3615 strain. The mixed starter of the OLL205013 strain and the OLS3290 strain had a time required for the second half of fermentation that is about 40 minutes longer, and in addition, had a time required for the first half of fermentation that is about 20 minutes shorter compared to the mixed starter of the OLL205013 strain and the OLS3615 strain. In particular, the mixed starter of the OLL205013 strain and the OLS3290 strain had a time required for the second half of fermentation of 294 minutes, which is longer than any other starters. Therefore, in the present invention, it can be said that using the mixed starter of the OLL205013 strain and the OLS3290 strain is optimal.

Furthermore, as shown in Tables 2 and 3, in general, the fermentation rate in the first half of fermentation can be increased and the decrease in pH in the second half of fermentation can be suppressed by using the OLL205013 strain as Lactobacillus bulgaricus. That is, in the production of a starter for fermented milk production in which Lactobacillus bulgaricus and Streptococcus thermophilus are mixed, when the OLL205013 strain was used as Lactobacillus bulgaricus, the decrease in pH in the second half of fermentation was suppressed while increasing the fermentation rate in the first half of fermentation, regardless of what strain was used as Streptococcus thermophilus to some extent. Therefore, it can be said that the OLL205013 strain is at the core of the characteristic effect of the present invention.

As shown in Table 2 and Table 3, in order to achieve the effects of the present invention, it is preferable that the OLL1222 strain or the OLL205013 strain be selected as Lactobacillus bulgaricus, and the OLS3290 strain or the OLS3615 strain be selected as Streptococcus thermophilus. As shown in Table 1, all of these Lactobacillus bulgaricus and Streptococcus thermophilus strains have a property that changes acidity of lactic acid of a skimmed milk powder medium containing 0.1 wt % of yeast extract to 0.8 or more and less than 1.0 when the strains are each purely cultured in the skimmed milk powder medium at 37° C. for 12 hours. In contrast, it was confirmed that the OLL1171 strain of Lactobacillus bulgaricus that was considered to be unsuitable in the present invention has a property that changes acidity of lactic acid of the medium to 1.0 when purely cultured under the same condition. Therefore, it is presumed that the effect of the present invention can be versatilely exhibited by using a mixed starter in which Lactobacillus bulgaricus and Streptococcus thermophilus that have a property that changes acidity of lactic acid of a medium to 0.8 or more and less than 1.0 (preferably 0.95) or less are combined under the same measurement conditions.

Furthermore, as shown in Table 1, all of Lactobacillus bulgaricus and Streptococcus thermophilus strains that are suitable for the use in the present invention have a property that changes pH of a skimmed milk powder medium containing 0.1 wt % of yeast extract to 4.1 to 4.6 when the strains are each purely cultured in the skimmed milk powder medium at 37° C. for 12 hours. In contrast, it was confirmed that the OLL1171 strain of Lactobacillus bulgaricus that was considered to be unsuitable in the present invention has a property that changes pH of a medium to 4.0 when purely cultured under the same condition. Therefore, it is presumed that the effect of the present invention can be versatilely exhibited by using a mixed starter in which Lactobacillus bulgaricus and Streptococcus thermophilus that have a property that changes pH of acidity of lactic acid of a medium to 4.1 to 4.6 (preferably 4.3 to 4.5) under the same measurement conditions.

### Other Comparative Example

Skimmed milk powder: 10 wt % and water: 90 wt % were mixed, heated to 95° C. (sterilization), and then cooled to 10° C. to prepare a yogurt base. Into the yogurt base, a commercially available starter (recommended addition rate) and LB 81 bulk starter (2 wt %) were inoculated and mixed, and then the mixture was dispensed to a test tube and fermented in a thermostat at 43° C. The time required for pH to reach 4.6 from the start of fermentation (fermentation time) and the time required for pH to decrease from 4.6 to 4.4 are shown in Table 4 below.

TABLE 4
The first half of fermentation: time required for pH to reach 4.60 (minute)

| | |
|---|---|
| So Greek S1 (Christian Hansen Holding A/S) | 330~360 |
| 2651 (Danisco A/S) | 330~360 |
| LB81 starter (Meiji Co., Ltd.) | 180~240 |

The second half of fermentation: time required for pH to decrease from
4.60 to 4.40 (minute)

| | |
|---|---|
| So Greek S1 (Christian Hansen Holding A/S) | 120 or less |
| 2651 (Danisco A/S) | 120 or less |
| LB81 starter (Meiji Co., Ltd.) | 60~120 |

As shown in Table 4, no lactic acid bacterium starter was found that has the time required for the second half of fermentation of 3 hours or more while maintaining the time required for the first half of fermentation within 9 hours.

### INDUSTRIAL APPLICABILITY

The present invention relates to a production method of a fermented milk such as yogurt. Therefore, the present invention can be suitably used in the manufacturing industry of a fermented milk such as yogurt.

1. A method for producing a fermented milk, comprising:
a step for adding a lactic acid bacterium starter to a raw material mix to obtain a fermented milk base material; and
a fermentation step for fermenting the fermented milk base material at 35° C. to 50° C.,
wherein, in the fermentation step, a time required for pH of the fermented milk base material to decrease from 4.6 to 4.4 is 3 hours or more.

2. The production method of a fermented milk according to claim 1,
wherein, in the fermentation step, a time required for pH of the fermented milk base material to reach 4.6 after addition of the lactic acid bacterium starter to the raw material mix is within 9 hours.

3. The production method according to claim 2,
wherein the lactic acid bacterium starter includes Lactobacillus bulgaricus that changes acidity of lactic acid of a skimmed milk powder medium containing 0.1 wt % of yeast extract to 0.8 or more and less than 1.0 when the Lactobacillus bulgaricus is purely cultured in the skimmed milk powder medium at 37° C. to 43° C. for 12 hours; and Streptococcus thermophilus that changes acidity of lactic acid of a skimmed milk powder medium containing 0.1 wt % of yeast extract to 0.8 or more and less than 1.0 when the Streptococcus thermophilus is purely cultured in the skimmed milk powder medium at 37° C. to 43° C. for 12 hours.

4. The production method according to claim 3,
wherein the Lactobacillus bulgaricus and the Streptococcus thermophilus is selected from a combination of a strain that decreases pH to 4.6 or less within 9 hours when the combination is subjected to mixed culture in a skimmed milk powder medium at 37° C. to 43° C.

5. The production method according to claim 1,
wherein the lactic acid bacterium starter includes a Lactobacillus delbrueckii subsp. bulgaricus OLL205013 strain (Accession No.: NITE BP-02411) and a Streptococcus thermophilus OLS3290 strain (Accession No.: FERM BP-19638) or OLS3615 strain (Accession No.: NITE BP-01696).

6. The production method according to claim 1,
wherein the lactic acid bacterium starter includes a Lactobacillus delbrueckii subsp. bulgaricus OLL205013 strain (Accession No.: NITE BP-02411) and a Streptococcus thermophilus OLS3290 (Accession No.: FERM BP-19638).

7. A Lactobacillus delbrueckii subsp. bulgaricus OLL205013 strain (Accession No.: NITE BP-02411).
